(19) United States
(12) Patent Application Publication
(10) Pub. No.: US 2017/0233712 A1
(43) Pub. Date: Aug. 17, 2017

(54) PROCESS FOR THE PREVENTION AND SUPPRESSION OF BACTERIAL DISEASES IN PLANTS

(71) Applicant: Ryan William Shepherd, Middleton, WI (US)

(72) Inventor: Ryan William Shepherd, Middleton, WI (US)

(73) Assignee: PhylloTech, LLC, Middleton, WI (US)

(21) Appl. No.: 15/428,380

(22) Filed: Feb. 9, 2017

Related U.S. Application Data
(60) Provisional application No. 62/294,077, filed on Feb. 11, 2016.

(51) Int. Cl. C12N 9/80; A01N 63/02; A01N 63/00; C12N 15/82

(52) U.S. Cl. CPC C12N 9/80; C12N 15/8281; A01N 63/02; A01N 63/00

(57) ABSTRACT

The invention is directed to methods and compositions that inhibit pathogen proliferation. More specifically, in various embodiments the present invention relates to activities of acylases as disruptors of bacterial disease, and thus virulence, and the utility of acylases as control agents for plant disease.

### CROSS-REFERENCE TO RELATED APPLICATIONS

This patent application claims priority under 35 U.S.C. §119 to U.S. Provisional Application Serial No. 62/294,077, filed Feb. 11, 2016, the disclosure of which is incorporated herein by reference.

### STATEMENT REGARDING FEDERALLY SPONSORED RESEARCH

The invention was supported by Grant 2015-33610-23784 from the United States Department of Agriculture, and therefore the government may have rights in the invention.

### REFERENCE TO SEQUENCE LISTINGS

This patent application references amino acid sequences provided in an electronic file. SEQ ID NO: 1, SEQ ID NO: 2, and SEQ ID NO: 3 are provided in the file Acylases.txt.

### BACKGROUND OF THE INVENTION

The need for new antimicrobial agents that prevent plant and animal diseases is growing as pathogens gain resistance to existing antimicrobials. In agriculture, available approaches for preventing seed and crop losses due to bacterial diseases are inadequate in many cases. Several approaches to control bacterial disesease are used currently, but success of those programs is inconsistent. Transgenic plants that attenuate bacterial diseases have been developed, but public acceptance of genetically modified food crops is a limitation of this strategy. Furthermore, the use of currently marketed topical sprays to control bacterial diseases have drawbacks, as many are thought to be hazardous to the environment, toxic to animals, or raise other public concerns. Thus, there is an immediate need to develop alternative control measures that do not have these perceived disadvantages to mitigate bacterial diseases in agriculture.

Methods of producing and using compositions useful as antimicrobial agents are provided. More specifically, in various embodiments the present invention relates to activities of acylases as disruptors of bacterial disease, and thus virulence, and the utility of acylases as control agents for plant disease.

### BRIEF SUMMARY OF THE INVENTION

This invention provides a family of acylases, or proteins produced by a bacteria, that can be used to control bacterial disease. Acylases are produced by many bacteria and vary in molecular size and other properties. Three acylases described herein were identified in the plant pathogens Pseudomonas syringae and Pseudomonas fluorescens. As described herein, acylases can suppress or abolish disease by P. syringae, Pectobacterium carotovorum and Dickeya dadantii, three pathogens belonging to the Gram-negative class of bacteria. Thus, acylase polypeptides of this invention suppress or abolish disease caused by bacteria.

This invention relates to acylase polypeptides, substantially purified acylase polypeptides and compositions comprising acylase polypeptides, particularly acylase polypeptides from pseudomonads, e.g. P. syringae and P. fluorescens, that have antimicrobial activity, particularly a broad spectrum anti-bacterial activity. Anti-bacterial as used herein refers to the suppression or abolishment of disease caused by bacteria, e.g., Gram-negative bacteria. In one aspect of this invention the acylase polypeptides, in their natural state, may have a molecular weight from about 60 kD to about 90 kD. The acylase polypeptides of this invention isolated from bacteria and compositions comprising the acylase polypeptides suppress or abolish disease caused by bacteria, preferably Gram-negative bacteria.

Also an aspect of this invention are compositions comprising the acylase polypeptides or substantially purified acylase polypeptides of this invention and variants or fragments thereof. Preferably the compositions of this invention comprise a polypeptide having the amino acid sequence set forth in SEQ ID NO: 1 (PssHacB). The compositions may also comprise a polypeptide having the amino acid set forth in SEQ ID NO: 2 (PssHacA) and SEQ ID NO: 3 (PfuHacB). The compositions may also comprise a variant of these acylase polypeptides or fragments thereof. The acylase polypeptides, substantially purified acylase polypeptides and compositions comprising acylase polypeptides or fragments or variants thereof having antimicrobial activity are suitable for suppressing or abolishing microbial growth on a subject, an organism or a surface that is susceptible to bacterial infection (e.g., plants and animals).

The acylase polypeptides, substantially purified acylase polypeptides and compositions comprising the acylase polypeptides of this invention, or fragments or variants thereof having antimicrobial activity, may be used to suppress microbial growth, particularly growth of a bacterial organism, on plants or their seeds, that are susceptible to infection by the microbes. Thus, also an aspect of this invention are compositions useful for treating plants that are susceptible to microbial infections wherein the compositions comprise proteins consisting essentially of acylases having antimicrobial, particularly anti-bacterial, activity. The plants may be treated prior to, or subsequently, to infection with the microbe to suppress or abolish progression of the disease.

A variant of the polypeptides of this invention may contain conservative substitutions of amino acids within the sequence, but is at least 80% identical, preferably greater than 80% identical, more preferably at least 90% identical and most preferably at least 95% identical, to SEQ ID NO:1, or to a fragment of SEQ ID NO:1, having antimicrobial activity, and is at least 50%, preferably at least 70%, more preferably at least 80% and most preferably at least 90% as effective as an equal molar amount of SEQ ID NO:1 in suppressing growth of a bacteria, on a subject, organism or surface, e.g., an animal or plant.

This invention also relates to an isolated nucleic acid molecule comprising the polynucleotide sequence encoding the polypeptides, or a homolog thereof with >80% identity preferably at least 90% identity and more preferably at least 95% identity. The invention further relates to a polypeptide encoded by the polynucleotide sequence or a homolog thereof with >80% identity, preferably at least 90% identity and more preferably at least 95% identity e.g., SEQ ID NO:1, that have antimicrobial activity.

The invention further relates to a method of suppressing microbial proliferation in or on a plant, e.g., by overexpression of an acylase gene in the plant, or contacting an infected plant with a acylase polypeptide, and in or on a surface by contacting the surface with an acylase polypeptide.

The invention further relates to plants selected from the group consisting of alfalfa, rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery, carrot, squash, pumpkin, Zucchini, cucumber, apple, pear, melon, strawberry, grape, raspberry, pineapple, soybean, tobacco, tomato, sorghum, sugarcane, and grasses, e.g., turf grasses and the like, genetically modified by a polypeptide of the invention.

The invention further relates to a method of inhibiting microbial proliferation in or on an organism comprising administering a therapeutically effective amount of an acylase polypeptide.

The invention provides acylase polypeptides, substantially pure acylase polypeptides and variants and fragments thereof, having antimicrobial activity, preferably anti-bacterial activity, and methods of using such polypeptides and compositions comprising such polypeptides, to enhance microbial, preferably bacterial, resistance in plants. In addition, the invention demonstrates that the acylase polypeptides of this invention are antimicrobial proteins useful in molecular farming products. Acylases have the potential to be used as inhibitors against human and animal microorganisms. Overexpression of acylase genes in plants such as corn, soybean, tobacco, tomato, potato, pepper, Datura, alfalfa, cucumber, medicago, vitis sp, grasses, e.g., turfgrass, and the like enhances plant resistance to bacterial microorganisms. In addition, the acylase polypeptides of this invention can be used as a topical bacterial inhibitor.

Other aspects of the invention are described throughout the specification.

### BRIEF DESCRIPTION OF THE DRAWINGS

FIG. 1. In potato tuber maceration assays, the exogenous addition of acylase (PssHacB) significantly decreased disease caused by Pectobacterium carotovorum and Dickeya dadantii.

FIG. 2. Potato tuber infection assay with Pcc. A) PBS; B) Pcc WPP14; C) wild-type P. fluorescens; D) P. fluorescens expressing acylase (PssHacB); E) Pcc WPP14+wild-type P. fluorescens; F) Pcc WPP14+ P. fluorescens expressing acylase (PssHacB).

FIG. 3. Bean pod infection assay with P. syringae. A) sterile water; B) P. syringae B728a mixed with purified acylase (PssHacB); C) P. syringae B728a alone. Black arrows denote internal tissue maceration.

### DETAILED DESCRIPTION OF THE INVENTION

The invention is directed to acylase polypeptides having antimicrobial, preferably antibacterial, activity and to compositions comprising the acylase polypeptides. The invention also relates to methods of using the acylase polypeptides to inhibit microbial, preferably bacterial, infection and disease of plants and animals, preferably humans, and to inhibit the germination and growth of bacteria in or on the surface of materials that are susceptible to infection.

### Definitions

To facilitate understanding of the invention set forth in the disclosure that follows, a number of terms are defined below.

The term “ Pseudomonas syringae” refers to a plant pathogen that naturally produces antibacterial compounds.

The term “acylase” refers to proteins produced by Pseudomonas that contribute to antibacterial activity.

The meaning of other terminology used herein should be easily understood by someone of ordinary skill in the art.

### Acylases

Acylases are expressed in bacteria such as pseudomonads. Typical biological activities or functions associated with this family of polypeptides, as described herein, include, e.g., suppression of bacterial disease. In one aspect of the invention acylase polypeptides include oligomers or fusion polypeptides comprising at least one domain portion of one or more acylase, or fragments of any of these acylases that have antimicrobial activity, and preferably are capable of suppressing or abolishing inhibiting bacterial disease.

This invention provides a family of polypeptides, termed acylases, and the utility of these polypeptides and homologous polypeptides (>80% homology, commonly >90% homology, more typically >95% homology) from other species as antimicrobials (e.g., antibacterials) against human and animal pathogens.

An acylase polypeptide of the invention includes a polypeptide that shares a sufficient degree of amino acid identity or similarity to a polypeptide having a sequence as set forth in SEQ ID NO:1, SEQ ID NO:2, or SEQ ID NO:3 such that it is likely to share particular structural domains, have biological activities in common with the acylase polypeptides of this invention, and/or bind to antibodies that also specifically bind to acylases comprising SEQ ID NO: 1, SEQ ID NO:2, and SEQ ID NO:3. The acylase polypeptides of the invention may be isolated from naturally occurring sources or they may be recombinantly produced and have the same structure as a naturally occurring acylase polypeptide, or may be produced to have structures that differ from naturally occurring acylases but retain a significant amount of antimicrobial activity. Polypeptides derived from any acylase polypeptide of the invention by any type of alteration (for example, but not limited to, insertions, deletions, or substitutions of amino acids, preferably conservative substitutions, changes in glycosylation of the polypeptide, refolding or isomerization to change its three-dimensional structure or self-association state, and changes to its association with other polypeptides or molecules) are also acylase polypeptides for the purposes of the invention. Therefore, the polypeptides provided by the invention include polypeptides characterized by amino acid sequences similar to those of the acylase polypeptides or similar to acylase polypeptides described herein, preferably a acylase comprising the amino acid sequences set forth in SEQ ID NO:1, SEQ ID NO: 2, and SEQ ID NO:3, but into which modifications are naturally provided or deliberately engineered. A polypeptide that shares biological activities in common with members of the acylase polypeptide family is a polypeptide having antimicrobial activity, preferably antibacterial activity.

Amino acid substitutions and other alterations (deletions, insertions, and the like) to the acylase amino acid sequences (e.g., SEQ ID NO:1, SEQ ID NO:2, or SEQ ID NO:3) that change the consensus residues of the amino acid sequences and particularly substitutions of an amino acid with one of dissimilar structure (e.g., such as substitution of any one of the aliphatic residues—Ala, Gly, Leu, Ile, or Val—with another non-aliphatic residue), or substitution or alteration of a residue that is conserved among acylases, are predicted to be more likely to alter or disrupt acylase polypeptide activities. Conversely, a substitution of a residue at a position in the alignment that is not conserved among acylase and acylase-like sequences, is less likely to affect the function of the altered acylase polypeptide. The invention provides acylase polypeptides and fragments of acylase polypeptides, comprising altered amino acid sequences. Altered acylase polypeptide sequences share at least 75% identity, preferably at least 85% to at least 95%, or most preferably at least 99%, identity with the acylase amino acid sequences set forth in SEQ ID NO:1, SEQ ID NO:2, or SEQ ID NO:3.

The invention provides both full-length and mature forms of acylase polypeptides. Particularly preferred “full-length” polypeptides are those having the complete amino acid sequence of the polypeptide as encoded by SEQ ID NO:1. The amino acid sequences of full-length polypeptides can be obtained, for example, by translation of the complete open reading frame (“ORF”) of a cDNA molecule. Several full-length polypeptides may be encoded by a single genetic locus if multiple mRNA forms are produced from that locus by alternative splicing or by the use of multiple translation initiation sites. An example of a full length polypeptide of the invention includes the sequence as set forth in SEQ ID NO:1. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide may be obtained by expression, in a suitable plant cell or other host cell, of a polynucleotide that encodes the full-length polypeptide.

The sequence of the mature form of the polypeptide may also be determinable from the amino acid sequence of the full-length form, through identification of signal sequences or protease cleavage sites. The acylase polypeptides of the invention also include those that result from post-transcriptional or post-translational processing, events such as alternate mRNA processing which can yield a truncated but biologically active polypeptide. Also encompassed within the invention are variations attributable to proteolysis such as differences in the N- or C-termini upon expression in different types of host cells, due to proteolytic removal of one or more terminal amino acids from the polypeptide (generally from about 1 to 5 terminal amino acids).

The invention further includes acylase polypeptides with or without associated native-pattern glycosylation. Polypeptides expressed in yeast or plant expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, typically provides non-glycosylated molecules. Further, a given preparation can include multiple differentially glycosylated species of the polypeptide. Glycosyl groups can be removed through conventional methods, in particular those utilizing glycopeptidase (Boehringer Mannheim).

Species homologues of acylase polypeptides and polynucleotides are also provided by the invention. As used herein, a “species homologue” is a polypeptide or polynucleotide with a different species of origin from that of a given polypeptide or polynucleotide, but with significant sequence similarity to the given polypeptide or polynucleotide. Species homologues may be isolated and identified by making suitable probes or primers from polynucleotides encoding the acylase polypeptides provided herein and screening a suitable nucleic acid source from the desired species. Alternatively, homologues may be identified by screening a genome database containing sequences from one or more species utilizing a sequence (e.g., nucleic acid or amino acid) of an acylase molecule of the invention. Such genome databases are readily available for a number of species. Computer algorithms, which connect two proteins through one or more intermediate sequences, can be used to identify closely related as well as distant homologs.

The invention also encompasses allelic variants of acylase polypeptides and polynucleotides; that is, naturally-occurring forms of such polypeptides and polynucleotides in which differences in amino acid or nucleotide sequence are attributable to genetic polymorphism.

Fragments of the acylase polypeptides of the invention are encompassed by the invention and may be in linear form or cyclized using known methods. acylase polypeptides and fragments thereof, and the polynucleotides encoding them, include amino acid or nucleotide sequence lengths that are at least 25% (typically at least 50%, 60%, 70%, and, most commonly at least 80%) of the length of an acylase polypeptide or polynucleotide and have at least 60% sequence identity (typically at least 70%, 75%, 80%, 85%, 90%, 95%, 97.5%, or at least 99%, and, most commonly at least 99.5%) with that acylase polypeptide or polynucleotide, where sequence identity is determined by comparing the amino acid or nucleotide sequences when aligned so as to maximize overlap and identity while minimizing sequence gaps. Methods for determining identity are discussed in more details below. Also included in the invention are polypeptides and fragments, and polynucleotides encoding them, that contain or encode a segment comprising at least 8, or at least 10, or at least 15, or typically at least 20, or still more typically at least 30, or most commonly at least 40 contiguous amino acids, preferably of SEQ ID No:1, SEQ ID NO:2, or SEQ ID NO:3. Such polypeptides and fragments may also contain a segment that shares at least 70% sequence identity (typically at least 75%, 80%, 85%, 90%, 95%, 97.5%, or at least 99%, and most commonly at least 99.5%) with any such segment of any of the acylase polypeptides or polynucleotides, where sequence identity is determined by comparing the sequences of the polypeptide or polynucleotide when aligned so as to maximize overlap and identity while minimizing sequence gaps.

The invention also provides for soluble forms of acylase polypeptides comprising certain fragments or domains of these polypeptides. Preferably the fragments or domains retain an acylase antimicrobial, preferably antibacterial, activity that is at least about 50%, 70%, 80% or 90% of the activity of the acylase providing the fragment or domain. Soluble polypeptides are polypeptides that are capable of being secreted from the cells in which they are expressed. Soluble acylase also include those polypeptides which include part of the transmembrane region, provided that the soluble acylase polypeptide is capable of being secreted from a cell, and typically retains acylase polypeptide activity. Soluble acylase polypeptides further include oligomers or fusion polypeptides comprising at least one acylase polypeptide and fragments of any of these polypeptides that have acylase polypeptide activity. A secreted soluble polypeptide may be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble acylase polypeptides are advantageous for many applications. Purification of the polypeptides from recombinant host cells is preferred, because soluble polypeptides are secreted from the cells and are generally more suitable than membrane-bound forms for parenteral administration.

In another aspect, the invention provides polypeptides comprising various combinations of polypeptide domains from different acylase polypeptides. In one embodiment, a fusion construct comprising at least one acylase domain are linked via a peptide linker.

This invention also relates to conservative variants of the acylases described herein, preferably conservative variants of a polypeptide having the amino acid sequence set forth in SEQ ID NO: 1, SEQ ID NO: 2, or SEQ ID NO:3. Conservative variants have conservative substitutions, as described below, of one or more amino acids. Preferably the conservative variants have amino acid lengths that are at least 25% (typically at least 50%, 60%, 70%, and, most commonly at least 80%) of the length of a acylase polypeptide or polynucleotide and have at least 60% sequence identity (typically at least 70%, 75%, 80%, 85%, 90%, 95%, 97.5%, or at least 99%, and, most commonly at least 99.5%) with that acylase polypeptide or polynucleotide. Those of skill in the art appreciate that certain amino acid residues may be substituted for other amino acid residues in a protein structure without appreciable loss of interactive capacity with structures such as, for example, substrate-binding regions. These changes are termed “conservative” in the sense that they preserve the structural and, presumably, required functional qualities of the starting molecule. Conservative amino acid residue substitutions generally are based on the relative similarity of the amino acid residue side-chain substituents, for example, their hydrophobicity, hydrophilicity, charge, size, and the like. An analysis of the size, shape and type of the amino acid residue side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine are defined herein as equivalent to each other; alanine, glycine and serine are defined herein as equivalent to each other; and phenylalanine, tryptophan and tyrosine are defined herein as equivalent to each.

In making such conservative substitutions, the hydropathic index of amino acid residues also may be considered. Each amino acid residue has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (-0.9); tyrosine (−1.3); proline (−1.6); histidine (−3.2); glutamate (−3.5); glutamine (−3.5); aspartate (-3.5); asparagine (−3.5); lysine (−3.9); and arginine (−4.5).

The importance of the hydropathic amino acid index in conferring interactive biological function on a protein is generally understood in the art. It is known that certain amino acid residues may be substituted for other amino acid residues having a similar hydropathic index or score and still retain a similar biological activity. In making changes based upon the hydropathic index, the substitution of amino acid residues whose hydropathic indices are within +/−2 is preferred, those which are within +/−1 are particularly preferred, and those within +/−0.5 are even more particularly preferred.

It also is understood in the art that conservative substitutions of like amino acid residues can be made effectively on the basis of hydrophilicity. The greatest local average hydrophilicity of a protein, as governed by the hydrophilicity of its adjacent amino acid residues, correlates with its immunogenicity and antigenicity, i.e., with a biological property of the protein. The following hydrophilicity values have been assigned to amino acid residues: arginine (+3.0); lysine (+3.0); aspartate (+3.0+/−1); glutamate (+3.0+/−1); serine (+0.3); asparagine (+0.2); glutamine (+0.2); glycine (0); threonine (−0.4); proline (−0.5+/−1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making conservative variants with substitutions based upon similar hydrophilicity values, the substitution of amino acid residues whose hydrophilicity values are within +/−2 is preferred, those which are within +/−1 are particularly preferred, and those within +/−0.5 are even more particularly preferred.

Additional variants within the scope of the invention include acylase polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (e.g., detectable) or therapeutic agents attached thereto are contemplated herein. Typically, such alteration, substitution, replacement, insertion or deletion retains the desired activity of the polypeptide or a substantial equivalent thereof.

Other derivatives include covalent or aggregative conjugates of the acylase with other polypeptides, such as by synthesis in recombinant culture as N-terminal or C-terminal fusion polypeptides. Examples of fusion polypeptides are discussed herein in connection with oligomers. Further, fusion polypeptides can comprise peptides added to facilitate purification and identification. Such peptides include, for example, poly-His or the antigenic identification peptides. One such peptide is the FLAG peptide, which is highly antigenic and provides an epitope reversibly bound by a specific monoclonal antibody, by enabling rapid assay and facile purification of the expressed recombinant polypeptide.

As used herein, a “chimeric polypeptide” or “fusion polypeptide” comprises a acylase (including fragments having antimicrobial, preferably anti-bacterial activity) polypeptide of the invention operatively linked to a second polypeptide. The second polypeptide can be any polypeptide of interest having an activity or function independent of or related to the function of a acylase polypeptide. For example, the second polypeptide can have a related activity to a acylase polypeptide and can be a domain of a related but distinct member of the acylase family of proteins such as, for example, cytoplasmic or transmembrane domain of a related acylase polypeptide. Within the fusion polypeptide, the term “operatively linked” is intended to indicate that a acylase polypeptide and the second polypeptide are fused in-frame to each other. The second polypeptide can be fused to the N-terminus or C-terminus of a acylase of the invention. Additional examples of polypeptides of interest include peptide linkers, Fc polypeptides, leucine zipper polypeptides, and the like.

Encompassed by the invention are oligomers or fusion polypeptides that contain a acylase polypeptide, one or more fragments of acylase polypeptides, or any of the derivative or variant forms thereof as disclosed herein. In particular embodiments, the oligomers comprise soluble acylase polypeptides. Oligomers can be in the form of covalently linked or non-covalently-linked multimers, including dimers, trimers, or higher oligomers. Leucine zippers and polypeptides derived from antibodies are among the peptides that can promote oligomerization of the polypeptides attached thereto.

In another aspect, a fusion polypeptide comprising multiple acylase polypeptides, with or without peptide linkers (spacer peptides) is provided. In some embodiments, a linker moiety separates the acylase polypeptide domain and the second polypeptide domain in a fusion polypeptide. Such linkers are operatively linked to the C- and the N-terminal amino acids, respectively, of the two polypeptides. Typically a linker will be a peptide linker moiety. The length of the linker moiety is chosen to optimize the biological activity of the soluble acylase and can be determined empirically without undue experimentation. The linker moiety should be long enough and flexible enough to allow a acylase moiety to freely interact with a substrate or ligand. The linker moiety is a peptide between about one and 30 amino acid residues in length, typically between about two and 15 amino acid residues. One linker moiety is a -Gly-Gly- linker. The linker moiety can include flexible spacer amino acid sequences, such as those known in single-chain antibody research. A DNA sequence encoding a desired peptide linker can be inserted between, and in the same reading frame as, the heterologous sequences (e.g., a acylase encoding nucleic acid) and a second polypeptide encoding nucleic acid, using any suitable conventional technique. For example, a chemically synthesized oligonucleotide encoding the linker can be ligated between the sequences encoding a acylase polypeptide and a second polypeptide of interest. In particular embodiments, a fusion polypeptide comprises from two to four soluble acylase polypeptides separated by peptide linkers.

A polypeptide of the invention may be prepared by culturing transformed and/or recombinant host cells under culture conditions suitable to express the recombinant polypeptide. The resulting expressed polypeptide may then be purified from such culture (i.e., from culture medium or cell extracts) using known purification processes, such as gel filtration and ion exchange chromatography. The purification of the polypeptide may also include an affinity column containing agents which will bind to the polypeptide; one or more column steps over such affinity resins as concanavalin A-agarose, Heparin-toyopearl.™. or Cibacrom blue 3GA Sepharose.™.; one or more steps involving hydrophobic interaction chromatography using such resins as phenyl ether, butyl ether, or propyl ether; or immunoaffinity chromatography. Alternatively, the polypeptide of the invention may be expressed in a form that will facilitate purification. For example, it may be expressed as a fusion polypeptide comprising, for example, maltose binding polypeptide (MBP), glutathione-5-transferase (GST) or thioredoxin (TRX). Kits for expression and purification of such fusion polypeptides are commercially available from New England BioLab (Beverly, Mass.), Pharmacia (Piscataway, N.J.) and InVitrogen, respectively. The polypeptide can also be tagged with an epitope and subsequently purified by using a specific antibody directed to such epitope. One such epitope (“FLAG.™.”) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can be employed to provide a substantially purified homogeneous recombinant polypeptide. A acylase polypeptide thus purified is substantially free of other polypeptides and is defined in accordance with the invention as a “substantially purified polypeptide”; such purified polypeptides of the invention include purified antibodies that bind to a acylase polypeptide, fragment, variant, binding partner and the like. An acylase polypeptide of the invention may also be expressed as a product of transgenic animals or plants, e.g., as a component of the milk of transgenic cows, goats, pigs, or sheep which are characterized by somatic or germ cells containing a polynucleotide encoding the acylase polypeptide of the invention.

It is also possible to utilize an affinity column comprising a polypeptide that binds an acylase polypeptide of the invention, such as a monoclonal antibody generated against an acylase polypeptide, to affinity-purify expressed polypeptides. Polypeptides can be removed from an affinity column using conventional techniques, e.g., in a high salt elution buffer and then dialyzed into a lower salt buffer or by changing pH or other components depending on the affinity matrix utilized, or be competitively removed using the naturally occurring substrate of the affinity moiety, such as a polypeptide derived from the invention. In this aspect of the invention, acylase-binding polypeptides, such as the anti-acylase antibodies of the invention or other polypeptides that can interact with a acylase polypeptide of the invention, can be bound to a solid phase support such as a column chromatography matrix or a similar substrate suitable for identifying, separating, or purifying expressed polypeptides of the invention. Adherence of binding polypeptides (e.g., antibodies) to a solid phase contacting surface can be accomplished by any means; for example, magnetic microspheres can be coated with these binding polypeptides and held in the incubation vessel through a magnetic field.

An acylase polypeptide may also be produced by known conventional chemical synthesis. Methods for constructing polypeptides by synthetic means are known in the art. The synthetically-constructed polypeptide, by virtue of sharing primary, secondary or tertiary structural and/or conformational characteristics with acylase polypeptides, may possess biological properties in common therewith, including antimicrobial activity. Thus, they may be employed as biologically active or immunological substitutes for natural, purified polypeptides in screening assays, the development of antibodies, and in treating microbial infections.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the art that multiple bands corresponding to the polypeptide can be visualized by SDS-PAGE, due to differential glycosylation, differential post-translational processing, and the like. In one aspect, the polypeptide of the invention is purified to substantial homogeneity, as indicated by a single polypeptide band upon analysis by SDS-PAGE. The polypeptide band can be visualized by silver staining, Coomassie blue staining, or by autoradiography.

Antibodies that are immunoreactive with an acylase polypeptide are provided herein. Such antibodies specifically bind to the polypeptide (e.g., a polypeptide consisting of SEQ ID NO:1, SEQ ID NO:2, SEQ ID NO:3, or fragments thereof) via the antigen-binding site of the antibody (as opposed to non-specific binding). In the invention, specifically binding acyalse antibodies are those that will specifically recognize and bind with acylase polypeptides, homologues, and variants, but not with other molecules. Similarly, specifically binding anti-acylase antibodies are those that will specifically recognize and bind with acylase polypeptides, homologues, and variants, but not with other molecules. In one embodiment, the antibodies are specific for a acylase polypeptide consisting of SEQ ID NO:1, SEQ ID NO:2, SEQ ID NO:3, or fragment thereof, and do not cross-react with other polypeptides including related acylase. In this manner, the acylase polypeptides, fragments, variants, fusion polypeptides, and the like, as set forth above can be employed as “immunogens” in producing antibodies immunoreactive therewith.

The antigenic determinants or epitopes of acylases used for immunization can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway et al., Immunobiology 3:9 (Garland Publishing Inc., 2nd ed. 1996)). Because folded polypeptides have complex surfaces, the number of epitopes available is quite numerous; however, due to the conformation of the polypeptide and steric hinderances, the number of antibodies that actually bind to the epitopes is less than the number of available epitopes (Janeway et al., supra). Epitopes can be identified by methods known in the art. Thus, one aspect of the invention relates to the antigenic epitopes of acylase polypeptides. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques known in the art such as solid-phase synthesis, chemical or enzymatic cleavage of a polypeptide, or using recombinant DNA technology.

Antigen-binding antibody fragments that recognize specific epitopes may be generated by known techniques. For example, such fragments include but are not limited to: the F(ab′).sub.2 fragments which can be produced by pepsin digestion of the antibody molecule and the Fab fragments which can be generated by reducing the disulfide bridges of the (ab′).sub.2 fragments. Alternatively, Fab expression libraries may be constructed to allow rapid and easy identification of monoclonal Fab fragments with the desired specificity. Techniques described for the production of single chain antibodies can also be adapted to produce single chain antibodies against acylase gene products. Single chain antibodies are formed by linking the heavy and light chain fragments of the Fv region via an amino acid bridge.

The terms “polynucleotide” as used herein, refers to a polymeric form of nucleotides of at least 10 bases in length (smaller nucleotide sequences are typically referred to as oligonucleotides). The nucleotides can be ribonucleotides, deoxyribonucleotides, or modified forms of either type of nucleotide. The term includes single and double stranded forms of DNA or RNA. DNA includes, for example, cDNA, genomic DNA, chemically synthesized DNA, DNA amplified by PCR, and combinations thereof. The polynucleotides of the invention include full-length genes or cDNA molecules as well as a combination of fragments thereof.

By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant polynucleotide molecule, which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

An acylase polynucleotide of the invention comprises: (a) a polynucleotide that encodes a polypeptide comprising a sequence set forth in SEQ ID NO:1, SEQ ID NO:2, and SEQ ID NO:3; and any of the foregoing wherein T can also be U (e.g., RNA sequences). Also encompassed by the invention are homologues of an acylase polynucleotide of the invention. Polynucleotide homologues can be identified in several ways, including isolation of genomic or cDNA molecules from a suitable source, or computer searches of available DNA sequence databases.

Polynucleotides encoding a polypeptide of this invention, or fragment thereof, or the complementary nucleotide sequence, can be used as probes or primers for the isolation of nucleic acids or as query sequences for database searches. Such probes or primers can be obtained by “back-translation” from the amino acid sequences, or by identification of regions of amino acid identity with polypeptides for which the coding DNA sequence has been identified. The polymerase chain reaction (PCR) procedure can be employed to isolate and amplify a polynucleotide encoding a acylase polypeptide or a desired combination of acylase polypeptide fragments. Oligonucleotides that define the desired termini of a combination of DNA fragments are employed as 5′ and 3′ primers. The oligonucleotides can additionally contain recognition sites for restriction endonucleases to facilitate insertion of the amplified DNA fragments into an expression vector.

Among the uses of the disclosed acylase polynucleotides, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. Using knowledge of the genetic code in combination with the amino acid sequences set forth above, sets of degenerate oligonucleotides can be prepared. Such oligonucleotides are useful as primers, e.g., in polymerase chain reactions (PCR). In certain embodiments, degenerate primers can be used as probes for non-human genetic libraries. Such libraries include, but are not limited to, cDNA libraries, genomic libraries, and even electronic EST (express sequence tag) or DNA libraries. Homologous sequences identified by this method would then be used as probes to identify acylase homologues.

The invention also includes polynucleotides that hybridize under moderately stringent conditions or highly stringent conditions, to polynucleotides encoding acylase polypeptides described herein. The basic parameters affecting the choice of hybridization conditions and guidance for devising suitable conditions can be readily determined by those having ordinary skill in the art based on, for example, the length and/or base composition of the DNA. One way of achieving moderately stringent conditions involves the use of a prewashing solution containing 5. times.SSC, 0.5% SDS, 1.0 mM EDTA (pH 8.0) at a temperature of about room temperature, and a hybridization buffer of about 50% formamide, 6.times.SSC, and a hybridization temperature of about 55.degree. C. (or other similar hybridization solutions, such as one containing about 50% formamide, with a hybridization temperature of about 42.degree. C.), and washing conditions of about 60.degree. C., in 0.5.times.SSC, 0.1% SDS. Generally, highly stringent conditions are defined as hybridization conditions as above, but with washing at approximately 68.degree. C., 0.2.times.SSC, 0.1% SDS. SSPE (1.times.SSPE is 0.15M NaCl, 10 mM NaH.sub.2PO.sub.4, and 1.25 mM EDTA, pH 7.4) can be substituted for SSC (1.times.SSC is 0.15M NaCl and 15 mM sodium citrate) in the hybridization and wash buffers; washes are performed for 15 minutes after hybridization is complete. The wash temperature and wash salt concentration can be adjusted as necessary to achieve a desired degree of stringency by applying the basic principles that govern hybridization reactions and duplex stability, as known to those skilled in the art and described further below. When hybridizing a nucleic acid to a target nucleic acid of unknown sequence, the hybrid length is assumed to be that of the hybridizing nucleic acid. When nucleic acid of known sequences are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10.degree. C. less than the melting temperature (T.sub.m) of the hybrid, where T.sub.m is determined according to the following equations. For hybrids less than 18 base pairs in length, T.sub.m (.degree. C.)=2(# of A+T bases)+4(# of G+C bases). For hybrids above 18 base pairs in length, T.degree. C.)=81.5+16.6(log [Na.sup.+])+0.41(% G+C)−(600/N), where N is the number of bases in the hybrid, and [Na.sup.+] is the concentration of sodium ions in the hybridization buffer ([Na.sup.+] for 1.times.SSC=0.165M). Each such hybridizing nucleic acid molecule has a length that is at least 15 nucleotides (or typically at least 18 to about 20 nucleotides, or at least 25 to about 30 nucleotides, or at least 40 nucleotides, or more commonly at least 50 nucleotides), or at least 25% (e.g., at least 50%, or at least 60%, or at least 70%, and most typically at least 80%) of the length of a polynucleotide of the invention to which it hybridizes, and has at least 60% sequence identity (e.g., at least 70% to about 75%, at least 80% to about 85%, at least 90% to about 95%, at least 97.5%, or at least 99%, and most commonly at least 99.5%) with a polynucleotide of the invention to which it hybridizes, where sequence identity is determined by comparing the sequences of the hybridizing nucleic acids when aligned so as to maximize overlap and identity while minimizing sequence gaps as described above.

The invention also provides genes corresponding to the polynucleotides disclosed herein. “Corresponding genes” are the regions of the genome that are transcribed to produce the mRNAs from which cDNA molecules are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated and includes both coding and non-coding regions.

Methods for making acylase polypeptides are described below. Expression, isolation, and purification of the polypeptides and fragments of the invention can be accomplished by any suitable technique, including but not limited to the following methods.

An isolated polynucleotide of the invention may be operably linked to an expression control sequence such as, e.g., the pDC412 or pDC314 vectors (Microbix Biosystems Inc., Toronto, Canada), pMal-cVx (BioRad), or the pMT2 or pED expression vectors, in order to produce an acylase polypeptide recombinantly. Many suitable expression control sequences are known in the art. As used herein “operably linked” means that a polynucleotide of the invention and an expression control sequence are situated within a construct, vector, or cell in such a way that the polypeptide encoded by a polynucleotide is expressed when appropriate molecules (such as polymerases) are present. In one embodiment, at least one expression control sequence is operably linked to an acylase polynucleotide of the invention in a recombinant host cell or progeny thereof, the polynucleotide and/or expression control sequence having been introduced into the host cell by transformation or transfection, for example, or by any other suitable method. In another embodiment, at least one expression control sequence is integrated into the genome of a recombinant host cell such that it is operably linked to a polynucleotide encoding an acylase polypeptide. In one embodiment of the invention, at least one expression control sequence is operably linked to a polynucleotide of the invention through the action of a trans-acting factor such as a transcription factor, either in vitro or in a recombinant host cell.

In addition, a polynucleotide encoding an appropriate signal peptide (native or heterologous) can be incorporated into expression vectors. The choice of signal sequence can depend on factors such as the type of host cells in which the recombinant polypeptide is to be produced. A DNA sequence for a signal sequence (secretory leader) can be fused in frame to a polynucleotide of the invention so that the DNA is initially transcribed, and the mRNA translated, into a fusion polypeptide comprising the signal peptide. A signal peptide that is functional in the intended host cells promotes secretion of the polypeptide. The signal peptide is cleaved from the polypeptide upon secretion of polypeptide from the cell. The skilled artisan will also recognize that the position(s) at which the signal peptide is cleaved can differ from that predicted by computer program, and can vary according to such factors as the type of host cells employed in expressing a recombinant polypeptide. A polypeptide preparation can include a mixture of polypeptide molecules having different N-terminal amino acids, resulting from cleavage of the signal peptide at more than one site. An acylase polypeptide of the invention may comprise a signal peptide from amino acid 1-25. This can be substituted by heterogenous signal peptides using known recombinant DNA techniques.

Established methods for introducing DNA into cells have been described. Additional protocols using commercially available reagents, such as Lipofectamine lipid reagent (Gibco/BRL) or Lipofectamine-Plus lipid reagent, can be used to transfect cells. Selection of stable transformants can be performed using methods known in the art such as, for example, resistance to cytotoxic drugs. A plasmid expressing the DHFR cDNA can be introduced into strain DX-B11, and only cells that contain the plasmid can grow in the appropriate selective media. Examples of selectable markers that can be incorporated into expression vectors include cDNAs conferring resistance to antibiotics, such as G418 and hygromycin B. Cells having the vector can be selected based on resistance to such compounds.

Alternatively, gene products can be obtained via homologous recombination, or “gene targeting” techniques. Such techniques employ the introduction of exogenous transcription control elements (such as the CMV promoter or the like) in a particular predetermined site on the genome, to induce expression of an endogenous acylase of the invention. The location of integration into a host chromosome or genome can be determined by one of skill in the art, given the known location and sequence of the gene. In one embodiment, the invention contemplates the introduction of exogenous transcriptional control elements in conjunction with an amplifiable gene, to produce increased amounts of the gene product.

A number of cell types may act as suitable host cells for expression of a polypeptide of the invention. It may be possible to produce the polypeptide in lower eukaryotes such as yeast or in prokaryotes such as bacteria, and in plant cells. Potentially suitable yeast strains include Saccharomyces cerevisiae, Schizosaccharomyces pombe, Kluyveromyces strains, Candida, or any yeast strain capable of expressing heterologous polypeptides. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments may be accomplished using known chemical or enzymatic methods. The polypeptides may also be produced by operably linking an isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac.™. kit). As used herein, a host cell capable of expressing a polynucleotide of the invention is “transformed.” Cell-free translation systems could also be employed to produce polypeptides using RNAs derived from polynucleotide constructs disclosed herein. A host cell that comprises an isolated polynucleotide of the invention, typically operably linked to at least one expression control sequence, is a “recombinant host cell”.

The acylase polypeptides, fragments (including soluble fragments), variants, antibodies, and binding partners of the invention are useful to improve the disease-resistance or disease-tolerance of plants either during the life of the plant or for post-harvest crop protection. Such polypeptides are also useful for suppressing or abolishing growth and proliferation of pathogens e.g.,bacteria, Pathogens exposed to such polypeptides are growth-inhibited. The antibacterial properties of an acylase can eradicate a pathogen already established on the plant or may protect the plant from future pathogen attack. The eradicant effect of the acylase polypeptides and fragments is particularly advantageous.

The acylases of this invention, e.g., acylases of pseudomonads, and compositions comprising the acylases can be used in methods to inhibit microbial growth and treat diseases, preferably diseases of plants caused by infection with pathogenic bacteria. Preferably the plants susceptible to disease caused by infection with the bacterial organism are crop plants, e.g., corn, soybean, tobacco, tomato, potato, pepper, Datura, alfalfa, cucumber, vitis sp and medicago, or grasses, e.g. turfgrasses, such as, e.g., annual and perennial rye grasses, and creeping bentgrass.

Exposure of a pathogen, e.g., a bacteria, to an acylase polypeptide can be achieved in various ways, for example: (a) The isolated acylase polypeptide may be applied to plant parts or to the soil or other growth medium surrounding the roots of the plants or to the seed of the plant before it is sown using standard agricultural techniques (such as, e.g., spraying). The acylase polypeptide may have been isolated from plant tissue or chemically synthesized or extracted from micro-organisms genetically modified to express the peptide. The acylase polypeptide may be applied to plants or to the plant growth medium in the form of a composition comprising the acylase polypeptide in admixture with a solid or liquid diluent and optionally various adjuvants such as surface-active agents. Solid compositions may be in the form of dispersible powders, granules, or grains. (b) A composition comprising a micro-organism genetically modified to express an acylase polypeptide may be applied to a plant or the soil in which a plant grows. (c) An endophyte genetically modified to express the acylase polypeptide may be introduced into the plant tissue (for example, via a seed treatment process). An endophyte is defined as a micro-organism having the ability to enter into non-pathogenic endosymbiotic relationships with a plant host. The endophyte may be genetically modified to produce agricultural chemicals. (d) DNA encoding an acylase polypeptide may be introduced into the plant genome so that the polypeptide is expressed within the plant body (the DNA may be cDNA, genomic DNA or DNA manufactured using a standard nucleic acid synthesizer).

In practicing a method of treatment or use of the invention, a therapeutically effective amount of a therapeutic agent of the invention is contacted with a plant, subject or surface to inhibit, treat or ameliorate a microbial (e.g., a bacterial) infection. “Therapeutic agent” includes without limitation any of the acylase polypeptides, fragments, and variants; soluble forms of the acylase polypeptides; antibodies to a acylase polypeptide or fragment; acylase polypeptide binding partners; complexes formed from the acylase polypeptides, fragments, variants, and binding partners, and the like. As used herein, the term “effective amount” or “therapeutically effective amount” means the total amount of each polypeptide or therapeutic agent or other active component of the pharmaceutical composition or method that is sufficient to show a meaningful benefit, e.g., treatment, healing, inhibition, prevention or amelioration of microbial contamination or infection, or an increase in rate of treatment, healing, inhibition, prevention or amelioration of such contamination and infections. Preferably the meaningful benefit is a statistically significant as compared to a control. Contacting a subject, organism or surface with the acylase polypeptides can be done in vitro or in vivo with an amount and for a time sufficient to reduce microbial infection or presence.

Compositions comprising a therapeutically effective amount of a acylase polypeptide, or variant, conservative variant, fragment, or oligomer thereof, (from whatever source derived, e.g., recombinant and non-recombinant sources), in combination with other components such as a physiologically acceptable diluent, carrier, or excipient, are provided herein and can be used in the methods described herein. The term “pharmaceutically acceptable” means a non-toxic material that does not interfere with the effectiveness of the biological activity of the active ingredient(s).

An acylase polypeptide of the invention (including fragments) may be active in multimers (e.g., heterodimers or homodimers) or complexes with itself or other polypeptides. As a result, pharmaceutical compositions of the invention may comprise a polypeptide of the invention in such multimeric or complexed form. Such compositions contemplate the preparation of fragments of acylase in any combination thereof as oligomers.

In yet another aspect, the invention provides methods for producing a transgenic plant which expresses a nucleic acid segment encoding the acylase protein of the invention. The process of producing transgenic plants is well-known in the art. In general, the method comprises transforming a suitable host cell, e.g., a corn, soybean, tobacco, tomato, potato, pepper, Datura, alfalfa, cucumber, medicago or grass, e.g., turfgrass, cell, with a DNA segment which contains a promoter operatively linked to a coding region that encodes an acylase protein. Such a coding region is generally operatively linked to a transcription-terminating region, whereby the promoter is capable of driving the transcription of the coding region in the cell, and hence providing the cell the ability to produce the recombinant protein in vivo. Alternatively, in instances where it is desirable to control, regulate, or decrease the amount of a particular recombinant acylase protein expressed in a particular transgenic cell, the invention also provides for the expression of acylase protein antisense mRNA. The use of antisense mRNA as a means of controlling or decreasing the amount of a given protein of interest in a cell is well-known in the art.

The invention further provides a transgenic plant, seed, cell, e.g., corn, soybean, tobacco, tomato, potato, pepper, Datura, alfalfa, cucumber, vitis sp, medicago or grass, e.g., turfgrass, plant, seed, cell, or any other form of regenerant, comprising a heterologous polynucleotide (>80% homology, commonly >90% homology, more typically >95% homology) selected from the group consisting of a) a polynucleotide comprising SEQ ID NO:4; b) a polynucleotide that hybridizes under moderate to highly stringent conditions to a polynucleotide comprising the sequence of SEQ ID NO:4 and encoding a polypeptide that is a disease- or pest-resistant conferring protein; c) a nucleotide sequence complementary to a sequence of SEQ ID NO:4; and d) any of the nucleotide sequences of a) to c) wherein T can also be U.

### EXAMPLES

### Example 1

### Control of Soft Rot with Acylase

Pectobacterium carotovorum and Dickeya dadantii cause bacterial soft rot diseases with a broad host range. Pathogenesis is mediated by a suite of cell wall degrading enzymes that cause tissue maceration and eventual rotting. To assess the ability of acylase strain to decrease potato soft rot disease, we performed tuber slice assays with the bacterial pathogens Pectobacterium carotovorum subsp. carotovorum (Pcc) strain WPP14 and Dickeya dadantii (Da) strain 3937.

Pcc WPP14 was grown in LB medium overnight. After washing cells in PBS (3×), the final concentration was adjusted to OD₆₀₀ of 0.4. Similarly, Escherichia coli expressing gfp and E. coli expressing acylase (PssHacB) strains were grown overnight in LB medium supplemented with kanamycin, subsequently washed with Phosphate Buffered Saline (PBS, 3×), and the final concentration was adjusted to OD₆₀₀ of 0.8. Pcc was then either mixed (1:1) with PBS or E. coli, yielding a final 0D₆₀₀ of 0.2 for Pcc and 0.4 for E. coli. For the tuber slice assay, potato tubers were surface-sterilized with 10% sodium hypochlorite (10 minutes), rinsed thoroughly, dried, and sliced into 0.5-0.6 cm thick sections. For each treatment, 12 tuber slices were inoculated with 10 μL of cell suspension. Sterile water or PBS were used as negative controls. The randomized tuber slices were inoculated, placed in humid plastic bins, and incubated at 28° C. for three days. To quantify soft rot, the amount of macerated tissue was collected and measured by weight (Table 1).

Under the test conditions employed, without Pcc, there was no evidence of soft rot disease—neither the negative control nor the E. coli plasmid strains (expressing gfp) displayed tuber rot symptoms. When E. coli expressing acylase (HacB) was co-incubated with Pcc WPP14, there was a significant reduction in maceration compared to Pcc alone.

TABLE 1
Development of maceration on potato tuber slices infected with
10 μL of Pcc, E. coli expressing green fluorescent protein
(gfp), E. coli expressing acylase (PssHacB), and buffer control.

| | Macerated potato tissue |
|---|---|
| Treatment | (grams)^{z} |
| Negative control (sterile | 0^{a} |
| PBS) | |
| Pcc WPP14 | 1.08 ± 0.14^{b} |
| E. coli gfp | 0^{a} |
| E. coli PssHacB | 0^{a} |
| E. coli gfp + Pcc WPP14 | 1.24 ± 0.26^{b} |
| E. coli PssHacB + Pcc | 0.60 ± 0.15^{c} |
| WPP14 | |

^{z}Means were calculated from macerated tissue weight of 16 tuber slices. Means with the same letter are not significantly different at P = 0.05. Error is standard error of the mean.

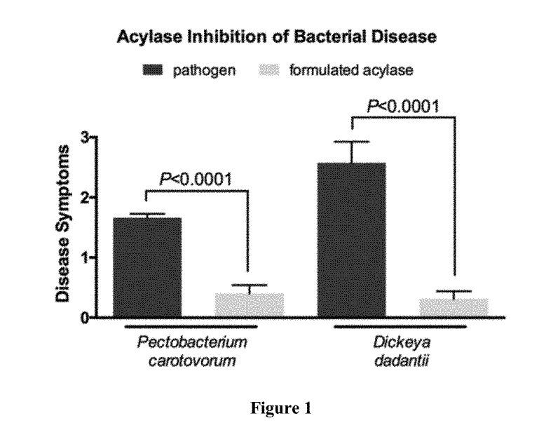

To further investigate the disease-suppression effects of the acylase PssHacB, we overexpressed PssHacB with a C-terminal histidine tag in P. syringae. The tag was then used to purify PssHacB, and potato maceration assays were performed with Pcc and Dd, as described above. Exogenous PssHacB significantly decreased the incidence of disease of both Pcc and Dd (FIG. 1).

### Example 2

### Control of Soft Rot with Acylase Produced by a Microbial Biocontrol Strain

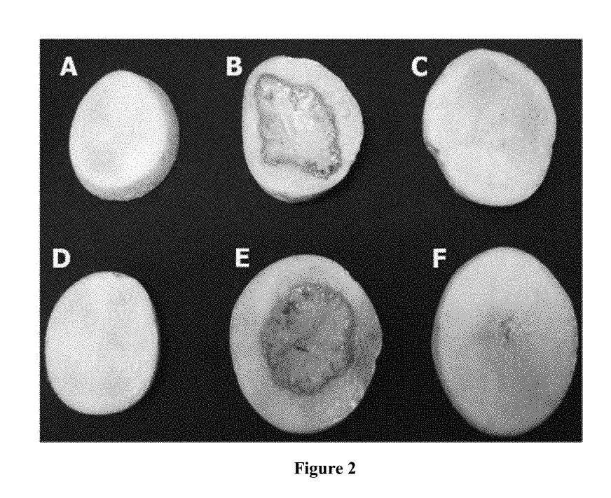

P. fluorescens strain A506 is a biocontrol strain used to suppress bacterial disease. We evaluated the capacity of a P. fluorescens constitutively expressing PfuHacB strain to limit soft rot. Similarly to the experiments with E. coli, Pcc and/or P. fluorescens were applied to potato tubers and incubated at 28° C. After incubation, the disease progression was quantified by collecting and weighing macerated tissue (FIG. 2). When P. fluorescens expressing PfuHacB was co-incubated with Pcc, there was significant reduction of potato maceration compared to Pcc alone or Pcc mixed with wild-type P. fluorescens (Table 2).

TABLE 2
Development of maceration on potato tuber slices infected
with Pcc, P. fluorescens A506, P. fluorescens A506
expressing acylase (PfuHacB), and buffer control.

| | Macerated potato tissue |
|---|---|
| Treatment | (grams)^{z} |
| Negative control (sterile | 0^{a} |
| water) | |
| Pcc WPP14 | 2.852 ± 0.42^{b} |
| P. fluorescens WT | 0^{a} |
| P. fluorescens PfuHacB | 0^{a} |
| P. fluorescens PfuHacB + | 1.867 ± 0.36^{c} |
| Pcc WPP14 | |

^{z}Means were calculated from macerated tissue weight of 12 tuber slices that were assayed in two independent experiments. Means with the same letter are not significantly different at P = 0.05. Error is standard error of the mean.

### Example 3

### Control of Brown Spot Disease with Acylase

P. syringae pv. syringae is a pathogen capable of causing brown spot disease in bean plants. To further investigate the disease-suppression effects of PssHacB, we overexpressed PssHacB with a C-terminal histidine tag in P. syringae. The tag was then used to purify PssHacB.

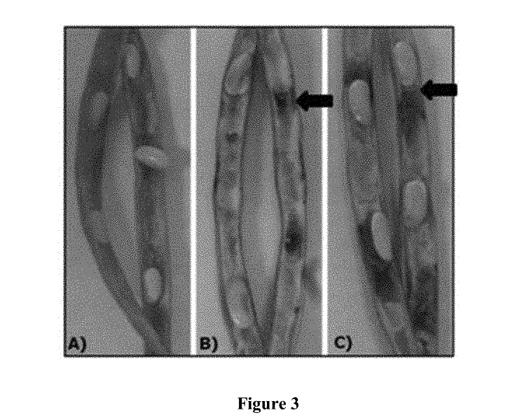

Suppression effects of the PssHacB enzyme on disease development by Pseudomonas syringae was assessed with bean pod inoculation assays. For each treatment, 12 Phaseolus vulgaris pods were surface sterilized with 10% sodium hypochlorite (one minute), rinsed thoroughly, and stabbed arbitrarily at two different sites on each pod using a sterile toothpick. Subsequently, 5 μL of P. syringae B728a cells (10⁹ CFU/mL) alone or in combination with purified PssHacB enzyme were injected into the stabbed site. Inoculated pods were incubated under humid conditions at 28° C. for 5 days. Disease symptoms were quantified by making longitudinal sections of the pod and measuring internal tissue maceration (Table 3). Significant differences in tissue maceration were observed among treatments. Bean pod treatment did not cause appreciable tissue discoloration. However, injection of P. syringae did cause brown spot formation. Co-inoculation of bean pods with P. syringae and purified PssHacB led to significantly reduced development of symptomatic disease (FIG. 3).

TABLE 3
Development of brown spot disease on bean pods infected with
5 μL of P. syringae, purified PssHacB enzyme, and sterile water

| | Internal discoloration |
|---|---|
| Treatment | (centimeters)^{z} |
| Negative control (sterile | 0.009 ± 0.05^{a} |
| water) | |
| P. syringae | 0.226 ± 0.022^{b} |
| P. syringae + PssHacB | 0.093 ± 0.15^{c} |

^{z}Means were calculated from internal discoloration lengths of 12 bean pods that were assayed in three independent experiments. Means with the same letter are not significantly different at P = 0.05. Error is standard error of the mean.

Although the preceding description contains significant detail, it should not be construed as limiting the scope of the invention but rather as a description of the embodiment of the preferred methods of the invention.

The examples set forth above are provided to give those of ordinary skill in the art with a complete disclosure and description of how to make and use the various embodiments of the invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference

What is claimed is:

1. A method for suppressing or abolishing disease and/or imparting disease resistance to plants comprising the application of an acylase in the presence or absence of another control to the cells of a plant, whereby disease is suppressed or abolished, or resistance to plant diseases is imparted to the plant.

2. A method according to claim 1, wherein the plant is selected from the group consisting of dicots and monocots.

3. A method according to claim 2, wherein the plant is selected from the group consisting of rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery, carrot, squash, pumpkin, zucchini, cucumber, apple, pear, melon, strawberry, grape, raspberry, pineapple, soybean, tobacco, tomato, sorghum, and sugarcane.

4. A method according to claim 1, wherein the acylase is administered to plants or plant growth medium in the form of a composition comprising the acylase polypeptide in admixture with a solid or liquid diluent and optionally various adjuvants such as surface-active agents.

5. A method according to claim 1, wherein the acylase is applied by spraying, soil drenching, irrigation, chemigation, broadcasting, in furrow, seed coating, or dip application.

6. A method according to claim 1, wherein the acylase is applied to plants as a composition further comprising a carrier.

7. A method according to claim 6, wherein the carrier is selected from the group consisting of water and other aqueous solutions.

8. A method according to claim 6, wherein the carrier is selected from the group consisting of a microbial biocontrol strain.

9. A method according to claim 8, wherein the carrier microbial biocontrol strains are selected from a group consisting of Pseudomonas, Bacillus, Agwbacterium, Lysobacter, Trichoderma, Paecilomyces, Gliocladium, Ampelomyces, Pythium, Metschnikowia, Chromobacterium, Penicillium, Coniothyrium, Chaetomium, Mywthecium, Aureobasidium, Pantoea, Burkholderia, Streptomyces, Variovorax, Pasteuria, Lactobacillus, Paenibacillus, Xanthomonas genera.

10. A method according to claim 1, wherein the acylase is derived from Pseudomonads.

11. A method for inhibiting proliferation of a microbe in or on a plant comprising overexpressing an acylase in the plant.

12. A plant according to claim 10, wherein the plant is selected from the group consisting of dicots and monocots.

13. A plant according to claim 11, wherein the plant is selected from the group consisting of of alfalfa, rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery, carrot, squash, pumpkin, Zucchini, cucumber, apple, pear, melon, strawberry, grape, raspberry, pineapple, soybean, tobacco, tomato, sorghum, sugarcane, and grasses, e.g., turf grasses.


SEQUENCE LISTING (3 sequences)

SEQ ID NO: 1 - protein, 795 aa, Pseudomonas syringae
MKRSLFILAIIIALLAGGAGWYVNSKLPVRDGEIAMSRLQAPVTLRYDERGVPHIRAENEADLYRALGYAHAQDRLFQMEMLRRLARGELAEVLGPNLLETDKLFRSLRIREHADAYVARQDKNTPTWKALEAYIDGINQYQDTHARPMEFDALGIPRRRFTPEDTVSIAGYLAYSFAAAFRTEPLLTYVRDQLGPEYLKVFDLDWHPDGMLDSKPALADADWKGLNQLAQLSHKALEDAGLPQFEGSNAWVISGSRTQSGKPILAGDPHIRFSVPSVWYEAQLSAPGFELYGHFPALNPFALLGHNMDFGWSLTMFQNDDVDLIAEKTNPDNPNQVWYHGQWVDMKRTEQQIAVKGQAPVTLTLLESPHGPIVNNVMGKNAGQAPIAMWWSFLVSANPVLDGFYEANRADTLDKMRSAAEKIQSPGLNIVWANAKGDIGWWAAAQLPIRPQGVNPTFILDGSTAQADKLGFYPFSANPHEENPARGYIVSANFQPLSPTGMQIPGYYNPAERGQELNRQLSDASVKWDLAASKALQLDTQTDYAPSILRPLIPVLRSVVSDPDELALVERLASWKGDYPVDSVGATLFNQFLFDLTEETFHDELGDAMFETLLSSRVMDAALPRLAADADSPWWNNRNSPHEESRANTVKVAWRASVSHLRSLYGTNPDEWVWGKAHTLTQGHPLGSQKPLDMIFNVGPYAAPGTHEVPNNLSSSIRPAPWPVGYGPSTRRLIDFADPAHSLGINPVGQSGVPFDKHYSDQAKAFVNGEYLPQRFSEKDVAEHTEGVLRLVPGA

SEQ ID NO: 2 - protein, 779 aa, Pseudmonas syringae
MIISRPLCSFVFAGLSFAVILPAQALVEPGNQAARAEIRRTGFGVPHIVAANERGLGYGIGYAYAQDNLCLLANEVVTVNGQRSRYFGPDKATLEQRNNMASDLLFQWLNTPQALADFWNAQPREIRQLMQGYVAGYNRSLAEQTTQGLPQPCAAEWVRPISTDDLLRLTRRLLVEGGVGQFAEALAGATPPAQQKPLQVDAQQAQALQLAAARNQRFALERGSNAVAIGRELSANGRGMLLANPHFPWGGGMRFYQMHLTIPGKLDVMGAALPGLPLINIGFNQHLAWSHTVDTSKHFTLHRLQLDPKDSTRYLLDGQSVAMGKQQVSVDVKQADGSLKSVPRIVYSSIFGPVVQWPGKLDWDSKFAFSLRDANLQNDRVLQQWYAMDKADSLKAFQDSVRKIQGIPWVNTLAVDAQGQALYMNLSVVPNVDAARLARCSDPRIGTELIVLDGSRSECNWEVSAEAAQAGIYPSSRQPQLLRTDFVQHSNDSAWMVNPAAPLQGFSPLISQDGQPLGQRARFALDRLESLKTAGKISVENLQAMVMDNEVYQAGQVLPDLLTFCASELGDDAARLAPLCAALKDWDGRADLNSGIGFVYFQKIMTSMQAVASRWRVAFDPQDPVHTPSGLAIENPSVATALRAAMLAAVDDVAKAGLPAGSKWGDIQVSSISGKQIPIHGGPAGLGVYNAMQTVAGKDGKREVVSGTSYLQVVTFDEQGPKAQGLLAFSESSNPQSAHSSDQTEAFSKKQWQALPFTEQQIKADPAYEVQVISEEPDR

SEQ ID NO: 3 - protein, 784 aa, Pseudomonas fluorescens
MKRVLQVFAVLIVLVALGAGWYVYSKQPTRQGTVTLAHLQGSVTVRYDDRGVPHIRAENEADLYRALGYVHAQDRLFQMEIMRRLARGELAEVLGPKLLETDKLFRSLRIRERALSYVEHMDPDSASSKALQAYLDGINQYQDSHASPMEFDVLGIAKRPFTAEDSISVAGYMAYSFAAAFRTEPVLTYVRDRLGSAYLNVFDLDWQPKGALNLAASDWQTLGAIAALSEQALADNGLPQFEGSNAWAVSGSHTQSGKPLLAGDPHIRFSVPSVWYEAQLSAPGFELYGYHNALVPVAFLGHNLDFGWSLTMFQNDDLDLIAEKVNPDNPNQVWYHDQWVDMSSSEQQIQVKGQAPVTLTLRRSPHGPIINDVLGENAGSTPIAMWWAFLDSENPILDGFYQLNRADTLAKARAAAAKVSAPGLNIVWANAKGDIGWWAAAQLPIRPAGVNPAFILDGSTTQADKLGFYPFSANPQEENPSRGYVVSANAQPASPTGMPIPGYYNLADRGQQLNAQLSDTSVKWDVTNSQALQLGTTTAYGPRLLAPLLPVLREVVKDPAQLKLVEQLANWKGDYPLDSTSATLFNQLLFNLSDATFHPKLGDALFKTLLGTRVIDAALPRLAASADSPWWNGNRADTVKLAWDNSLTHLKATFGDDPAQWQWGKAHTLTHGHPLGLQKPLDKIFNVGPFPAPGSHEVPNNQTSPIGPAPWPVTYGPSTRRLIDFADPAHALTINPVGQSGVPFDTHYGDQAQSYIEGRYEQAHFSEEEVTANTRGTLKLLPAR